(19) United States
(12) Patent Application Publication
(10) Pub. No.: US 2017/0296298 A1
(43) Pub. Date: Oct. 19, 2017

(54) APPARATUS AND METHODS FOR PREVENTING OR REDUCING REPETITIVE USE INJURIES IN THE OPERATING ROOM

(71) Applicant: Michael J. Sillers, Birmingham, AL (US)

(72) Inventor: Michael J. Sillers, Birmingham, AL (US)

(21) Appl. No.: 15/098,492

(22) Filed: Apr. 14, 2016

(51) Int. Cl. A61B 90/60

(52) U.S. Cl. CPC A61B 90/60

(57) ABSTRACT

The present disclosure relates to ergonomic platforms and components, and methods for making and using such devices. The present invention also relates generally to medical devices and methods for use in the operating room by a clinician. In particular, the present invention encompasses platform devices and attendant features for preventing and/or reducing repetitive use injuries by surgeons, and methods for making and using such technologies.

### CROSS-REFERENCE TO RELATED APPLICATIONS

This application claims priority to U.S. Provisional Patent Application Ser. No. 62/147,189, filed Apr. 14, 2015, which is incorporated by reference in its entirety.

### FIELD OF THE INVENTION

The present invention relates generally to medical devices and methods for use in the operating room by a clinician. In particular, the present invention encompasses platform devices and attendant features for preventing and/or reducing repetitive use injuries by surgeons, and methods for making and using such technologies.

### BACKGROUND OF THE INVENTION

The following description is provided to assist the understanding of the reader. None of the information provided or references cited is admitted to be prior art.

Physical injuries to the neck and back constitute one of the most frequently encountered ergonomic afflictions by endoscopic surgeons and clinicians in general. While great care is taken to protect the patient in the operating room, e.g., multiple patient identification verifications are made, careful attention is paid to patient positioning to avoid potentially harmful pressure points, and the entire operating room (“O.R.”) staff participates in the “time out” to ensure that the correct procedure is being performed on the correct patient, very little attention is paid to ergonomic considerations of the surgeon. Recent publications have documented the frequency of repetitive use injuries, particularly among those utilizing laparoscopes or endoscopes to assist in their procedures. It is estimated that over a million laparoscopic cholecystectomies and nearly 500,000 endoscopic nasal and sinus surgeries are performed annually in the U.S., which portends the likelihood that such complaints among surgeons will only increase in the near future.

### SUMMARY OF THE INVENTION

In one aspect, the present invention provides for an adjustable ergonomic platform, comprising: (a) the adjustable platform; (b) a platform surface configured to conform to a body surface that defines a target region, wherein the surface is substantially contacted by one or more regions of a user's body; (c) one or more conduits capable of cooperatively interacting with the adjustable platform when each component is substantially aligned; and (d) one or more securement fixtures, wherein the one or more securement fixtures are configured to reversibly engaged the one or more one or more conduits in an engaged configuration, and wherein the one or more securement fixtures are configured to reversibly disengaged the one or more conduits in a disengaged configuration. In illustrative embodiments, the adjustable ergonomic platform further entails one or more of drapes, power generating pedals, venting components and/or padding.

In suitable embodiment, the conduit is in an orthogonal position relative to the platform. In some embodiments, the conduit is a vertical rail, wherein the platform is capable of sliding along the rail when engaged. In illustrative embodiments, the platform is engaged with the conduit at a locus when the conduit is in a vertical position, and wherein the securement fixture is engaged with the conduit at a second locus when the conduit is in a vertical positions. In some embodiments, the platform surface is composed of a material selected from the group consisting of silicone, rubber, vinyl, non-water hardenable urethane, plastic, plastic-based materials, fiberglass, metal, foam, ceramic, one or more polymers, resin, plaster, cellulosic materials, silicone-based materials, elastomeric materials and/or conformable (contour fitting or adjusting) materials, or any combination thereof.

In illustrative embodiments, the target region is a body surface is selected from the group consisting of one or both forearms, one or both arms, an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof. In illustrative embodiments, the platform has a shape selected from the group consisting of circular, rectangular, square, polygonal, curved, concentric, concave, perimetric, diamond, hexagonal, or triangular configurations, or any combination thereof. In illustrative embodiments, the platform and/or platform surface allows for treatment, medicament application, topical cream application, application of ointments, cold compresses application, application of ice, coolant application, or for applying heat, or any combination thereof.

In some embodiments, the height of the adjustable ergonomic platform is about from 0.01 meters (m) to about 10 m. The one or more conduits and/or the securement fixtures are composed of silicone, rubber, vinyl, non-water hardenable urethane, plastic, plastic-based materials, fiberglass, metal, ceramic, one or more polymers, resin, plaster, cellulosic materials, silicone-based materials, foam, elastomeric materials and/or conformable (contour fitting or adjusting) materials or any combination thereof, in suitable embodiments. In some embodiments, the platform and/or the platform surface allows for ventilation, moisture release, liquid drainage, or drying, or any combination thereof.

In illustrative embodiments, the one or more securement fixtures are selected from the group consisting of plugs, screws, caps, covers, seals, corks, rivets, and stoppers, or any combination thereof. In illustrative embodiments, the conduit and/or the one or more securement fixtures interact with the platform and/or one or more conduits in a manner selected from the group consisting of angled, straight, slanted, tapered, orthogonal, polygonal, curved, diagonal, random, or any combination thereof. In suitable embodiments, the platform, platform surface, one or more conduits and/or securement fixtures are removable or interchangeable, or both.

In certain embodiments, the platform surface is patterned, texturized, smooth, gripping, designable, modifiable, or any combination thereof. The adjustable ergonomic platform further comprises a repository in certain embodiments. In some embodiments, the one or more platforms, platform surfaces, conduits, securement fixtures or any combination thereof have a shape selected from the group consisting of angled, straight, slanted, tapered, curved, diagonal, random, polygonal, rectangular, square, circular, curved, concentric, concave, perimetric, diamond, hexagonal, or triangular configurations, or any combination thereof.

In illustrative embodiments, the platform and/or platform surface possesses adherences nodes. The platform is configured as a single unit is some embodiments, while in other embodiments, the platform is composed of one or more segment components that are reversibly secured to form the platform. In certain embodiments, the one or more segment components are joined by an interphase socket. In some embodiments, the interphase socket is an interlocking ball and socket that functions to swivel in any direction. In illustrative embodiments, the ball and socket cooperatively interact. The cooperative interaction is selected from the group consisting of one or more snapping components, screws, clamps, adhesives, locks, rivets, friction fitting, or any combination thereof, in some embodiments. In certain embodiments, the reversible engagement is selected from the group consisting of one or more snapping components, screws, clamps, adhesives, rivets, locks, friction fitting, or any combination thereof. In some embodiments, the securing mechanism of the securement fixture is selected from the group consisting of one or more snapping components, screws, clamps, adhesives, locks, friction fitting, rivets, or any combination thereof.

In one aspect, the present invention entails a method of preventing or reducing the occurrence of repetitive use injuries during a medical procedure, comprising: (a) providing an adjustable platform, wherein the adjustable platform is composed of a platform surface configured to conform to a body surface that defines a target region, wherein the surface is substantially contacted by one or more regions of a user's body; (b) adjusting the platform to a desired height by: (i) aligning the adjustable platform with a conduit, wherein the aligning allows for a cooperative interaction when substantially aligned; and securing one or more securement fixtures to the conduit, wherein the one or more securement fixtures are configured to reversibly engaged the one or more one or more conduits in an engaged configuration, and wherein the one or more securement fixtures are configured to reversibly disengaged the one or more conduits in a disengaged configuration; and (c) performing the medical procedure.

In illustrative embodiments, the medical procedure is selected from the group consisting of endoscopy, laparoscopy, surgery, minimally invasive surgery, and invasive surgery. In illustrative embodiments, the present technology is specifically designed for endoscopic nasal and sinus surgeries. In other embodiments, the technology of the present disclosure is configured to assist other otolaryngic procedures (ear and laryngeal surgery) as well as general, gynecologic and urologic laparoscopic procedures, among others. In illustrative embodiments, the methods further include draping the adjustable platform. In some embodiments, the steps of aligning and/or securing comprises engaging the platform with the conduit at a locus when the conduit is in a vertical position, and wherein the securement fixture is engaged with the conduit at a second locus when the conduit is in a vertical positions. In illustrative embodiments, the desired height is based on the patient's positioning and the height of the surgeon performing the operation. In illustrative embodiments, the methods or adjustable ergonomic platforms of the present invention provide for a securement fixture that allows for attachment to the bed or operating table of the patient undergoing the procedure.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 is a perspective view of an adjustable ergonomic platform of the present invention. FIGS. 1A, 1B, and 1C respectively show a top view, front view and side view of the adjustable ergonomic platform.

FIG. 2 is a detailed perspective view of an adjustable ergonomic platform of the present invention with a silicone pad secured to the top section of the arm rest.

FIG. 3 is a photograph taken in an operating room with an adjustable ergonomic platform attached to the base-rail guard of the patient bed. FIG. 3A shows an embodiment of the present invention as mounted at a specific height. FIG. 3B shows an embodiment of the present invention with silicone pad and sanitary draping.

FIG. 4 is a photograph taken in an operating room with an adjustable ergonomic platform in use by a surgeon performing laparoscopy.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

As used herein, unless otherwise stated, the singular forms “a,” “an,” and “the” include plural reference. Thus, for example, a reference to “a drape” or “the drape” includes a plurality of drapes.

As used herein, the term “about” will be understood by persons of ordinary skill in the art and will vary to some extent depending upon the context in which it is used. If there are uses of the term which are not clear to persons of ordinary skill in the art, given the context in which it is used, the term “about” in reference to quantitative values will mean up to plus or minus 10% of the enumerated value.

As used herein, the terms “body surface” or “target region,” or “resting region,” refer to a part or structure of the body or device, having a certain function or functions, which can provide an area for ergonomic improvement, and includes, but is not limited to, an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof.

As used herein, the terms “platform”, “arm rest”, “forearm rest”, and/or “endoscopic arm rest” used in the context of preparation, production, and/or methods of making a suitable surface for ameliorating musculoskeletal injuries or symptoms in surgeons by providing an adjustable substrate for a surgeon to rest a particular region or regions of her or his anatomy, e.g., a body surface or region and any ancillary regions coterminous therewith. Such platforms have various contemplated surfaces and padding features, and/or are composed of materials, which include, but are not limited to, silicone, rubber, vinyl, non-water hardenable urethane, plastic, plastic-based materials, fiberglass, metal, ceramic, polymers, resin, plaster, padding, polymers, copolymers, and the like.

As used herein, the terms “comparable” or “corresponding” in the context of comparing two or more outcomes, responses, or conditions refer to the same individual, response, treatment, etc. used in the comparison. In some embodiments, comparable features may be obtained from the same individual at different times. In other embodiments, comparable samples may be obtained from different individuals. In general, comparable variables are normalized by a common factor for control purposes.

As used herein, the term “composition” refers to a product or component of a larger device with specified materials in the specified amounts, as well as any product or component associated therewith.

As used herein, the terms “cooperatively interact” or “cooperatively interacting” refer to the association of two or more adjoining components, where each component functions to facilitate the association. For example, an adjustable or fitted rail and/or attachment would cooperatively interact with the component that the rail and/or attachment was fabricated to engage.

As used herein, the terms “disengage”, “reversibly disengage”, “reversibly disengaged”, and “disengaged configuration”, all refer to the act or state of no longer being securely associated or connected. For example, two components are disengaged with each other they are not in contact with each other. However, such components can be in contact while concomitantly occupying a disengaged state. In this circumstance, the components would not be securely engaged by such means as, for example, a locking mechanism. If such components are “reversibly disengaged” then the components are capable of engaging at a different time.

As used herein, any or all components and/or steps of the present invention may interact, connect, engage, lock, secure, rivet and/or disconnect, disengage, unlock, and/or remove from, for example, any other component or step of the present invention. Any or all components and/or steps of the present invention may be configured to accesses a port, portal, region, or complementary fitting, provided in some embodiments. In accord, any or all components and/or steps of the present invention may be positioned in in a manner selected from angled, straight, slanted, tapered, curved, diagonal, random, polygonal, rectangular, square, circular, curved, concentric, concave, perimetric, diamond, hexagonal, or triangular configurations, or any combination thereof, capable of reversibly interacting, engaging, connecting, locking, and/or securing to any or all components and/or steps of the present invention in suitable embodiments. Moreover, any or all components and/or steps of the present invention may be configured as, e.g., hollow or solid rods, poles, members, tubes, plugs, screws, caps, rivets, covers, keys, wrenches, gripping members, and the like, or any combination thereof.

As used herein, the terms “engage”, “reversible engage”, “reversibly engaged”, and “engaged configuration” all refer to the act or state of being associated or connected in a secure manner for the purpose of joining two or more components for a period of time. For example, two components are engaged with each other when they are in contact and securely connected or associated for a period of time. To be in the engaged state, the components are in contact while concomitantly occupying an engaged state, such as, for example, a locked state. If such components are “reversibly engaged” then the components can be engaged and disengaged with respect to the features enabling such association and disassociation, respectively.

As used herein, the term “reticulated material” refers to compositions or composition matrices composed of network constituents forming one or more layers or matrix configurations. For example, reticulated material include, but are not limited to, fiberglass, silicone, one or more polymers, plastic, resin, plaster, and padding, or any combination thereof.

As used herein, the terms “substantial” or “substantially” within the context of “substantial contact” to a surface or region, or a “substantially aligned” configuration, refer to, e.g., total or complete contact or alignment, and the like, but also includes lesser than complete or total contact or alignment, and the like, insofar as the intended purpose for performing the act can be carried out to the same extent as if the, e.g., contact or alignment, were total or complete.

As used herein, the terms “treating” or “treatment” or “alleviation” refer to both therapeutic treatment and prophylactic or preventative measures, where the objective is to prevent or slow down (lessen) the targeted injury, condition or disorder. A subject, patient or medical clinician is successfully “treated” for a disorder if, after receiving therapeutic intervention according to the methods of the present invention, she or he shows observable and/or measurable reduction in or absence of one or more signs and symptoms of a particular injury or condition.

### Overview

Without question, great care is taken to protect the patient in the operating room. Multiple patient identification verifications are made, careful attention is paid to patient positioning to avoid potentially harmful pressure points, and the entire O.R. staff participates in a rest period/time-out to ensure that the correct procedure is being performed on the correct patient. However, very little attention is paid to ergonomic considerations of the surgeon and recent publications have documented the frequency of repetitive use injuries, particularly among those utilizing laparoscopes or endoscopes to assist in their procedures. Recent publications have documented the frequency of repetitive use injuries, particularly among those utilizing laparoscopes or endoscopes to assist in their procedures. It is estimated that over a million laparoscopic cholecystectomies and nearly 500,000 endoscopic nasal and sinus surgeries are performed annually in the U.S., which portends the likelihood that such complaints among surgeons will only increase in the near future.

In recent years, moreover, the roles of medical and surgical practitioners have undergone a major transformation, owing to developments in a new generation of advanced technologies such as surgical robotics, in-theatre interactive three dimensional displays, speech recognition for the control of critical theatre systems, virtual reality simulators, telemedicine, and e-learning. Yet despite the research and development community's enthusiasm for innovation, the end users—practitioners and specialists—are often ignored during the design and development processes, sometimes with serious consequences. The present disclosure addresses and remedies this ever-increasing problem as follows.

Accordingly, the present invention relates to, inter alia, novel adjustable ergonomic platform devices, components, systems and methods, which constitute an entire device or part of an entire device for alleviating repetitive use injuries by surgeons. Likewise, the present invention also includes methods for making and using such devices and apparatuses to allow, facilitate, enhance, and/or improve the process of endoscopic surgery, laparoscopic surgery or surgery in general by, in part, improving and optimizing the ergonomical conditions for the surgeon. Such aspects of the present invention consequently provide for increased comfort during surgery, decreased associated repetitive use injuries and therefore related procedures to treat such injuries, which may keep a surgeon from being able to perform in the O.R.

Surgeons and various medical practitioners, when performing surgeries and other medical procedures in the O.R. may develop and consequently suffer from acute musculoskeletal conditions, such as, but not limited to, muscular asymmetry, muscular atrophy, muscular degeneration, muscular denervation, and/or muscular denervation atrophy, and the like. Complications stemming from the eventual lack of flexibility and related fine motor skills can be career threatening. Along the same lines, excessive and repetitive use indications can lead to chronic pain, joint stiffness, muscle atrophy, or more severe complications, e.g., complex regional pain syndrome. The present invention, having the commercial name “EndoRest™” remedies these indications.

Impetus for correcting repetitive use injuries in surgical ergonomics emanates, in part, from the breadth of knowledge surrounding various sports-related injuries. Such injuries can be exacerbated by poor surgeon related O.R. ergonomics. For example, many surgeons begin presenting signs of back, neck, and shoulder pain, etc., following many years of performing surgical procedures. Indeed, the present inventor became afflicted with such indications after approximately fifteen years in an academic and private otolaryngology-head and neck surgery practice. It is not atypical for the pain associated with repetitive use injuries to escalate to the point of requiring medical imaging diagnostics, e.g., to ascertain the extend of the damage. In severe cases, bulging cervical discs may be identified as the ergonomic—etiological—agent, which in many instances requires subsequent medical intervention and time lost from work.

Inasmuch as improper surgical ergonomics is postulated as an underlying cause for repetitive use injuries in the O.R., confirmation by a professional in the field of medical ergonomics may be beneficial in some embodiments. In this regard, observation of patient positioning, the stance and posture of the surgeon, time allotted for breaks during surgery, if possible, and the general position of personnel and equipment in the O.R. may provide insight as to potential remediation that may improve O.R. ergonomics. In certain embodiments, ergonomic assessment tools, such as the Rapid Entire Body Assessment (“REBA”) is employed to systematically process and evaluate whole body postural MSD and risks associated with various tasks, which, in the present context, comprises repetitive use motions in the O.R. Even with such improvements, however, repetitive use injuries may remain.

Along these lines, the present invention is based on, and incorporates, various precept-components of medical ergonomics. Some of these components and end-point objectives include, but are not limited to, optimizing system performance while maximizing human wellbeing and operational effectiveness; equipment or systems design and training, which relate to body size (anthropometry), motion, and strength capabilities (biomechanics) of the end user, e.g., which in the present context includes surgeons; sensory-motor capabilities—vision, hearing, haptics (force and touch), and dexterity; cognitive processes and memory (including situational awareness) as well as muscle memory; training and knowledge relating to various O.R. equipment, systems, and practices as well as training associated with various medical conditions being treated; the expectations and cultural stereotypes relating to the operation of equipment; and the general health, age, motivation, stress levels, mental fatigue, and performance under various empirical circumstances. See, e.g., Stone, R. “Ergonomics in medicine and surgery.” BMJ. 2004 May 8; 328(7448): 1115-1118.

In this respect, however, the present invention endeavors to go beyond traditional or conventional ergonomic remediation. Previously, it has been suggested that such endeavors will only achieve success when implemented at the foundational level, where entire design settings in the O.R. will be required to undergo a systemic reconfiguration. See Stone, R. “Ergonomics in medicine and surgery.” BMJ. 2004 May 8; 328(7448): 1115-1118. It has been stated that designing an operating room of the future, which optimizes developments in surgery, will requires much more than plugging in the new technologies, where an approach must focus on re-designs at a systems level. Id.

Marked improvements in medical/surgical ergonomics, however, do not necessarily impart the manufacture of complex and/or intricate structures, nor do they require a complete redesign of the O.R. Indeed, a prototype of the present invention entailed an arm/shoulder rest composed of copper piping, duct tape and sheet metal. Subsequent to several refinements and iterative modifications, the present invention was a working manufacture that was readily integrated into the O.R. setting, e.g., during endoscopic nasal and sinus procedures, among other procedures. In some embodiments, the design of the present technology provides for a decrease in severity, or prevention, of shoulder fatigue while eliminating attendant pain associated with such fatigue. In some embodiments, the present invention provides for reducing, eliminating, lessening, and the like, of one or more of the following conditions selected from the group consisting of fatigue or pain in, on or around the head, neck, shoulder, wrists, forearms, elbows, trunk, back, legs and knees, among other body regions, in some embodiments.

More specifically, the simplicity of design of the present technology allows it to fit into or attach onto a standard O.R. table rail clamp, while also possessing capabilities allowing for adjustable height and angle settings depending on variables relating to, but not limited to, surgeon height, patient position in general, and the head position of the patient in particular. In some embodiments these factors are associated with planned procedure methods that can be taken into account prior to any particular surgery. A variety of disposable drapes, moreover, have been manufactured and configured specifically for the device of the present invention in illustrative embodiments. In suitable embodiments, these drapes are clear, transparent, opaque, colored, lightweight, removable, modifiable, and/or uniform in composition and size, among other metric configurations. In some embodiments, the draping is clear so that a variety of foot pedals can be visualized (electrocautery, scope cleaners, powered instrumentation). Illustrative embodiments of the present invention, as shown in FIG. 2, include padding features 120 to prevent paresthesias. In suitable embodiments, the padding 120 is composed of foam, silicone, rubber, vinyl, non-water hardenable urethane, plastic, plastic-based materials, fiberglass, metal, foam, ceramic, one or more polymers, resin, plaster, cellulosic materials, silicone-based materials, elastomeric materials and/or conformable (contour fitting or adjusting) materials, or any combination thereof.

In illustrative embodiments, the medical procedure is selected from the group consisting of endoscopy, laparoscopy, surgery, minimally invasive surgery, and invasive surgery. In illustrative embodiments, the present technology is specifically designed for endoscopic nasal and sinus surgeries. In other embodiments, the technology of the present disclosure is configured to assist other otolaryngic procedures (ear and laryngeal surgery) as well as general, gynecologic and urologic laparoscopic procedures, among others.

### Components, Applications, and Related Features

The present invention provides for an adjustable ergonomic platform, comprising: (a) the adjustable platform; (b) a platform surface configured to conform to a body surface that defines a target region, wherein the surface is substantially contacted by one or more regions of a user's body; (c) one or more conduits capable of cooperatively interacting with the adjustable platform when each component is substantially aligned; and (d) one or more securement fixtures, wherein the one or more securement fixtures are configured to reversibly engaged the one or more one or more conduits in an engaged configuration, and wherein the one or more securement fixtures are configured to reversibly disengaged the one or more conduits in a disengaged configuration. In illustrative embodiments, the adjustable ergonomic platform further entails one or more of drapes, power generating pedals, venting components and/or padding.

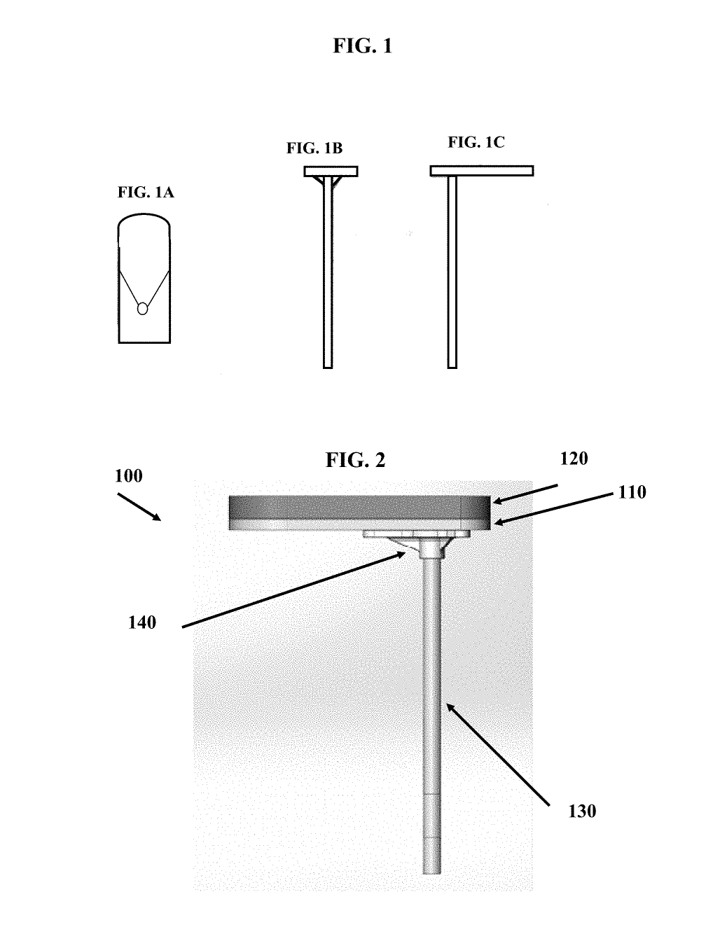

In one aspect, the present invention entails an adjustable ergonomic device design that fits into or onto a standard O.R. table rail clamp and has adjustable height and angle depending on surgeon height, patient position in general, head position in particular and planned procedure. See FIGS. 1-4. Along these lines, the platform component or arm rest section of the present technology is designed as a platform structure to allow the user to place their upper forearm and elbow to rest during a surgical procedure. In this regard, the upper surface of the platform is preferably relatively smooth and free from surface irregularities that might irritate the user's arm or that may make cleaning the surface difficult. Along the same lines, in suitable embodiment, the adjustable ergonomic device further incorporates padding to prevent, for example, paresthesias.

In this regard, the platform is configured as a single unit is some embodiments, while in other embodiments, the platform is composed of one or more segment components that are reversibly secured to form the platform. In certain embodiments, the one or more segment components are joined by an interphase socket. In some embodiments, the interphase socket is an interlocking ball and socket joint that functions to swivel in any direction thereby aiding the user in ergonomic mobility. In illustrative embodiments, the ball and socket cooperatively interact. The cooperative interaction is selected from the group consisting of one or more snapping components, screws, clamps, adhesives, locks, rivets, friction fitting, joint attachments, e.g., ball and socket joints, or any combination thereof, in some embodiments. In certain embodiments, the reversible engagement is selected from the group consisting of one or more snapping components, screws, clamps, adhesives, rivets, locks, friction fitting, or any combination thereof. In some embodiments, the securing mechanism of the securement fixture is selected from the group consisting of one or more snapping components, screws, clamps, adhesives, locks, friction fitting, rivets, or any combination thereof.

Within this purview, a pivoting mechanism allows the platform, via the ball and socket joint, to be angled relative to the user and/or conduit, between 0 and 180 degrees to support the surgeon's arm at whatever angle the surgeon wishes, or by swiveling out the surgeon's way entirely. This range includes all values and subranges therebetween. The platform may also be configured to angle up or down relative to the plane of the stretcher using a sliding collar adjustment in some embodiments. The platform may also be contoured to cradle the user's arm or target region contacting the platform surface. Optionally attached to the platform, platform surface and/or the padding attached thereto, is, depending on use preference, either a VELCRO-type hook and loop fastener or a latching strap to provide further restraint or support on the platform. In some embodiments, all swiveling, sliding, attaching, cooperatively interacting, secured and/or and folding connections between structural elements are separated by ultra-low coefficient of friction virgin polytetrafluoroethylene (or Teflon®) or ultra-high-molecular-weight (UHMW) polyethylene spacers to prevent aluminum self-galling and seizing of mating surfaces, incorporate oil-impregnated bronze bushings, and are held together with stainless steel bolts and deformed-thread self-locking nuts tightened to prevent unwanted movement.

While the angulation of the platform arm-board to the ground and/or conduit connecting the components of the present device is adjustable by the platform's own mechanism, all other motions are possible, and can be varied, namely: the position of the platform arm-board along the length of the operating table; the vertical inclination of the platform arm-board; the height of the platform arm-board; and the rotation of the platform arm-board along its longitudinal axis. All such positions are adjustable at the socket device interface, and once selected, are all simultaneously and in a single step, secured by tightening the torque bar of the socket mechanism.

In accord with embodiments of the present invention, moreover, the padding 120 and/or matting application may be applied to the arm rest portion 110 of the invention or any other component section, or combinations of sections as assembled, that is subject to contact from the surgeon during a procedure. In this respect, various layers of materials that function to absorb pressure from the contacting body region of the surgeon and/or provide frictional stability to decrease the possibility of slipping are included in the design assembly or as add-on features. Adequate padding 120 at the olecranon, ulnar styloid, and antecubital fossa, furthermore, may be required to decrease shock and discomfort of the contacting region, as noted in FIG. 2.

Likewise, the padding is applied to any feature of the present technology insofar as it aids in reducing unwanted forces, vibrations, and other tactile effects (shock, pressure point distress, etc.) of the body region contacting the adjustable ergonomic device. See FIGS. 2 and 4B. In some embodiments, the padding 120 is composed of foam, silicone, rubber, vinyl, non-water hardenable urethane, plastic, plastic-based materials, fiberglass, metal, foam, ceramic, one or more polymers, resin, plaster, cellulosic materials, silicone-based materials, elastomeric materials and/or conformable (contour fitting or adjusting) materials, or any combination thereof. In illustrative embodiments, the padding 120 is connected, secured, fixed, attached and the like, to the arm rest section of the platform 110 and may be configured as a single component or a multicomponent shock absorbing feature in accord with the platform configuration 100.

A variety of disposable drapes made specifically for the device are components of some embodiments, where the drapes provide for a sterile device inasmuch as they are removable and intended for a single use, i.e., one surgical procedure. In illustrative embodiments, the drape is composed of a clear material so that a visual sight-line is not impeded when considering the various instrument or device features that a surgeon must see in clear view during a surgical procedure, e.g., foot pedals which must be visualized (electrocautery, scope cleaners, powered instrumentation).

As shown in FIG. 1, and more fully detailed in the following sections, the conduit and securement fixtures of the present technology function as removably attaching features that support the platform. These features are lightweight yet sufficiently rigid to resist flexing and vibration during use. In come embodiments, the conduit and securement features are composed of carbon fiber, silicone, rubber, vinyl, non-water hardenable urethane, plastic, plastic-based materials, fiberglass, metal, foam, ceramic, one or more polymers, resin, plaster, cellulosic materials, silicone-based materials, elastomeric materials and/or conformable (contour fitting or adjusting) materials, or any combination thereof.

### System and Design Features

In one aspect, the present invention provides an adjustable ergonomic platform system 100 as shown in FIG. 2, where the platform 110 is capable of being adjusted in any direction in some embodiments. In illustrative embodiments, the surgeon or any personnel working under the direction of the surgeon is able to adjust the elevation of the platform to a desired height. Likewise, the tilt, latitude, incline, decline, angle, rotational stability, and the like, are adjustable in certain embodiments. Adjusting the platform, or any other feature of the present invention, is performed by varying the module, e.g., securement fixture 140, that functions to secure the platform at a desired height. In some embodiments, a securement fixture 140 or module component is a snapping components, screws, clamps, adhesives, locks, rivets, friction fitting, or any combination thereof. Thus, in some embodiments, the module component is loosened or tightened to vary the height of the platform, while is other embodiments, the module is engaged or disengaged to allow for the adjustment. In suitable embodiments, the height of the adjustable ergonomic platform is about from 0.01 meters (m) to about 10 m.

In illustrative embodiments, the height and/or width of the one or more adjustable platforms (as a whole or each constituent thereof, individually) is from about 0.001, 0.01, 0.1, 0.5, 0.75, 1, 2, 3, 4, or 5 meters (m) to about from 0.01, 0.1, 0.5, 0.75, 1, 2, 3, 4, 5, 6, 7, 8, 9, 10, 15, 20, 25 or 30 m. More specifically, some embodiments of the present invention provide for platforms having a height and/or width from about 0.1, 0.5, 0.75, or 1 m to about from 0.5, 0.75, 1, 2, or 3 m. In suitable embodiments, the height and/or width of the one or more platforms is from about 0.01 m to about 10 m, while in illustrative embodiments, the height is from about 2 m to about 4 m.

The platform including the platform surface 110, in some embodiments, is configured to conform to a body surface that defines a target region, where the surface is substantially contacted by one or more regions of a user's, i.e., the surgeon's, body. In this regard, the target region is a body surface is selected from the group consisting of one or both forearms, one or both arms, an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof. In illustrative embodiments, the target region, as shown in FIG. 4, is the forearm of the surgeon, which contacts the platform surface.

In some embodiments, the platform surface 110 is composed of a material selected from the group consisting of silicone, rubber, vinyl, non-water hardenable urethane, plastic, plastic-based materials, fiberglass, metal, Teflon, foam, ceramic, one or more polymers, resin, plaster, cellulosic materials, silicone-based materials, elastomeric materials and/or conformable (contour fitting or adjusting) materials, or any combination thereof. The shape of the platform region, moreover, is designed to conform to the contours of the surgeons arm, thus eliminating or reducing unwanted forces, vibrations, and other tactile effects (shock, pressure point distress, etc.) of the target body region contacting the platform surface 110 of the adjustable ergonomic device. See FIGS. 2 and 4B.

The shape of the platform 110, in selected embodiments, includes, but is not limited to, circular, oval, rectangular, square, polygonal, curved, concentric, concave, perimetric, diamond, hexagonal, or triangular configurations, or any combination thereof. In illustrative embodiments, the platform and/or platform surface allows for treatment, medicament application, topical cream application, application of ointments, cold compresses application, application of ice, coolant application, or for applying heat, or any combination thereof, insofar as such features may be required by the surgeon.

In illustrative embodiments, the platform surface 110 is patterned, texturized, smooth, gripping, designable, modifiable, or any combination thereof. The adjustable ergonomic platform further comprises a repository in certain embodiments. In some embodiments, the one or more platforms 110, platform surfaces 110, conduits 130, securement fixtures 140 or any combination thereof as shown in FIG. 2, have a shape selected from the group consisting of angled, straight, slanted, tapered, curved, diagonal, random, polygonal, rectangular, square, circular, curved, concentric, concave, perimetric, diamond, hexagonal, or triangular configurations, or any combination thereof.

In suitable embodiments, the platform and/or platform surface possesses adherences nodes. The platform is configured as a single unit is some embodiments, while in other embodiments, the platform is composed of one or more segment components that are reversibly secured to form the platform. In certain embodiments, the one or more segment components are joined by an interphase socket. In some embodiments, the interphase socket is an interlocking ball and socket that functions to swivel in any direction. In illustrative embodiments, the ball and socket cooperatively interact. The cooperative interaction is selected from the group consisting of one or more snapping components, screws, clamps, adhesives, locks, rivets, friction fitting, or any combination thereof, in some embodiments. In certain embodiments, the reversible engagement is selected from the group consisting of one or more snapping components, screws, clamps, adhesives, rivets, locks, friction fitting, or any combination thereof. In some embodiments, the securing mechanism of the securement fixture is selected from the group consisting of one or more snapping components, screws, clamps, adhesives, locks, friction fitting, rivets, or any combination thereof.

The systems of the present technology further entail one or more conduits 130, e.g., poles, rods, supporting barrels, posts, tubes and the like, which may be configured as, e.g., hollow, partially hollow, or solid structures, or any combination thereof, all of which are capable of cooperatively interacting with the adjustable platform when each component is substantially aligned. See FIGS. 1-4. This interaction allows for the adjustable features to be secured in a desired position, while also allowing for further adjustments to be made should the position of the surgeon require system modification. In some embodiments, the conduit is in an orthogonal position relative to the platform. In some embodiments, the conduit is a vertical rail, wherein the platform is capable of sliding along the rail when engaged. In illustrative embodiments, the platform 110 is engaged with the conduit 130 at a locus when the conduit 130 is in a vertical position, and wherein the securement fixture 140 is engaged with the conduit at a second locus when the conduit 130 is in a vertical positions.

The one or more conduits 130 and/or the securement fixtures 140 are composed of carbon fiber, silicone, rubber, vinyl, non-water hardenable urethane, plastic, plastic-based materials, fiberglass, metal, ceramic, one or more polymers, resin, plaster, cellulosic materials, silicone-based materials, foam, elastomeric materials and/or conformable (contour fitting or adjusting) materials or any combination thereof, in suitable embodiments. In some embodiments, the platform 110 and/or the platform surface 110 and/or 120 allows for ventilation, moisture release, liquid drainage, or drying, or any combination thereof.

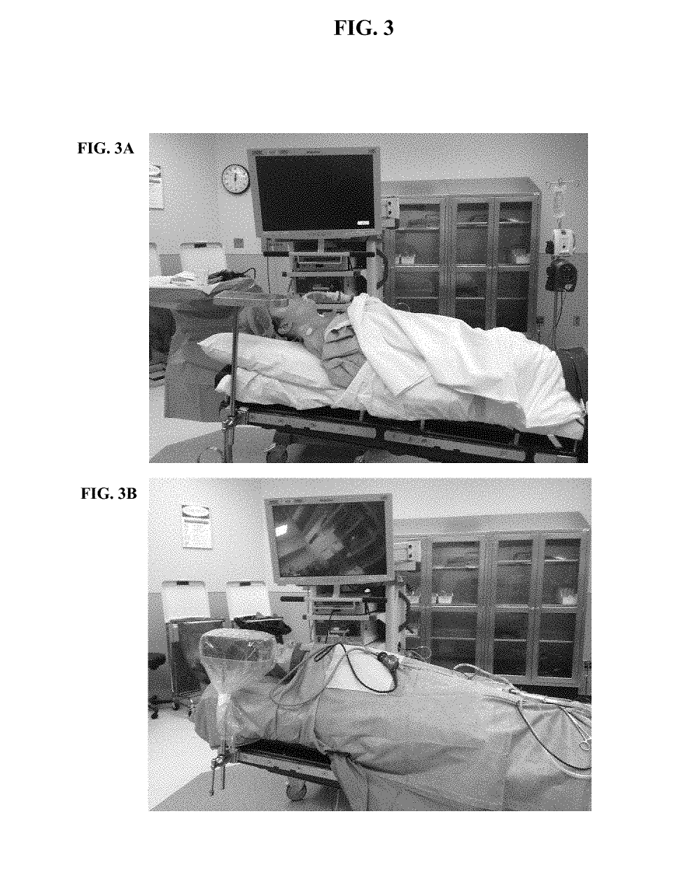

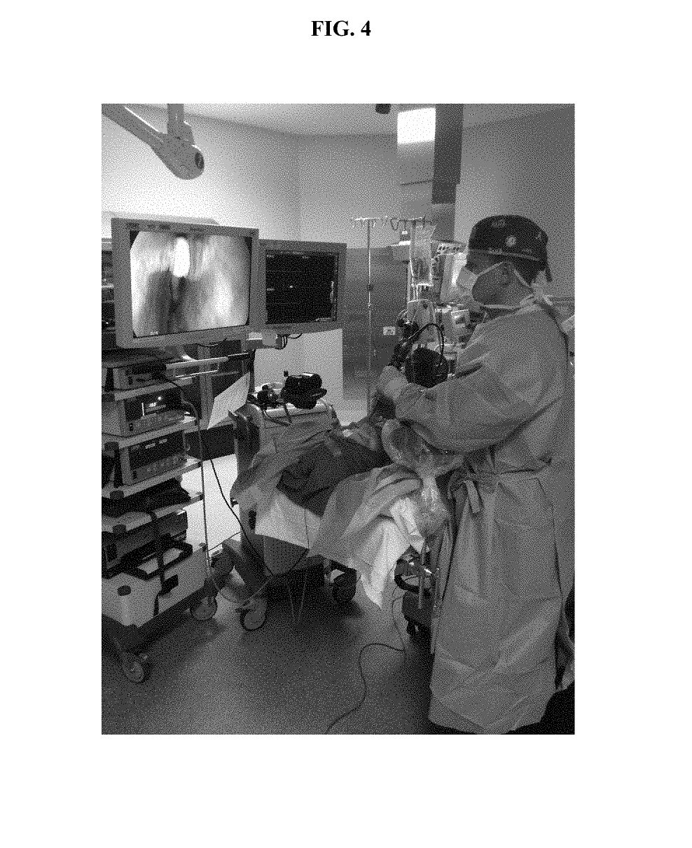

In illustrative embodiments, the one or more securement fixtures 140 are selected from the group consisting of plugs, screws, caps, covers, seals, corks, rivets, and stoppers, or any combination thereof. In illustrative embodiments, the conduit 130 and/or the one or more securement fixtures 140 interact with the platform and/or one or more conduits 130 in a manner selected from the group consisting of angled, straight, slanted, tapered, orthogonal, polygonal, curved, diagonal, random, or any combination thereof. In suitable embodiments, the platform 110, platform 110 and/or 120 surface, one or more conduits 130 and/or securement fixtures 140 are removable or interchangeable, or both. In suitable embodiments, one or more securement fixtures 140, wherein the one or more securement fixtures 140 are configured to reversibly engaged the one or more one or more conduits 130 in an engaged configuration, and wherein the one or more securement fixtures 140 are configured to reversibly disengaged the one or more conduits 130 in a disengaged configuration. In illustrative embodiments, the adjustable ergonomic platform further entails one or more of drapes (see FIG. 3A), power generating pedals, venting components and/or padding.

The present invention also provides for a device where any or all components of the present invention are capable of cooperatively interacting with any or all components of the present invention when substantially aligned, and a securing mechanism to reversibly connect any or all components of the present invention, and/or to reversibly connect any or all components of the present invention, where any or all components of the present invention are configured to reversibly engaged any or all components of the present invention in an engaged configuration, and where any or all components of the present invention are configured to reversibly disengaged any or all components of the present invention in a disengaged configuration.

In illustrative embodiments, any or all components of the present invention comprises one or more vents, half-cylinder “cylindania” and/or channels, which, in some embodiments, are position throughout any or all components of the present invention. Such vents allow for adequate ventilation and decreased moisture, e.g., from perspiration or humidity within an operating room, in certain embodiments. Accordingly, embodiments of the present invention as shown in FIG. 2 of the platform 110, surface platform 110 and/or padding feature 120 attendant thereto possess in particular embodiments, vents, channels and the like, to ensure that the target region of the surgeon—the body region contacting the platform, platform surface and/or the padding feature—is adequately secure and positioned, e.g., in the absence of slipping or unwanted movement. Likewise, any or all components of the present invention allow for treatment applications, medicaments, topical creams, ointments, cold compresses, ice, coolants, or heat, or any combination thereof, which may be required for a surgeon performing the operation. The vents, cylindania and/or channels can be of any shape or size to the extent that such shapes and sizes provide for the intended purpose, as described herein. In some embodiments, the shape of the vents, cylindania and/or channels are selected from conical, frusto-conical, rounded, flat, pointed, burred, bubble-shaped, dome-shaped, cylindrical, half-cylinder, tubed, circular, pen-circular, rectangular, square, polygonal, curved, concentric, concave, perimetric, diamond, hexagonal, or triangular configurations, or any combination thereof.

While any or all components of the present invention are typically positioned on the surface of the platform 110 and/or conduit 130, the cylindania are borne out of the inferior region of any or all component surfaces of the present invention, which imparts a means of increased air circulation and target body region stability. The cylindania have shapes selected from cylindrical, half-cylinder, tubed, peri-circular, rectangular, square, polygonal, curved, concentric, concave, perimetric, diamond, hexagonal, or triangular configurations, or any combination thereof. Conduits 130 and/or platforms 110 of the present invention further include adherence nodes protruding from the inferior region of the lower conduit surface and/or any other region. Such nodes directly contact a body surface while at least functioning to secure, anchor, position and/or adhere any or all components to the target region.

In concert with the conduit features detailed above, any or all components of the present invention are configured and/or manufactured as single component sections, combined sectional components, e.g., connecting silicone bridges in suitable embodiments, and/or as an entire structure via additive manufacturing processes such as, for example, 3D printing. Any or all components of the present invention interact, connect, engage, and/or disconnect, disengage, and/or remove from the conduits as necessary to impart a functional structure for the intended uses described herein. In some embodiments, any or all components of the present invention, which relate to connecting or securing mechanisms are selected from, e.g., plugs, screws, caps, covers, seals, corks, and stoppers, or any combination thereof. Any or all components of the present invention may be composed of silicone, rubber, adhesive, foam, TPE, vinyl, non-water hardenable urethane, plastic materials, fiberglass, metal, ceramic, polymers, resin, plaster, cellulosic materials, foam or any combination thereof. Any or all components of the present invention may have shapes and sizes are provided in suitable embodiments, and include, but are not limited to, for example, shapes such as, e.g., polygonal, rectangular, square, circular, curved, concentric, concave, perimetric, diamond, hexagonal, or triangular configurations, or any combination thereof, such that any or all components of the present invention are capable of cooperatively interacting, interacting, engaging, and/or connecting to or covering the open region of a conduit.

In some embodiments, any or all components of the present invention interact with any or all components of the present invention in a manner selected from angled, straight, slanted, tapered, orthogonal, polygonal, curved, diagonal, random, or any combination thereof. Such interaction may occur via plugging, screwing, capping, covering, sealing, corking, locking, riveting, and/or stopping, or any combination thereof. In illustrative embodiments, any or all components of the present invention are removable or interchangeable, or both.

In suitable embodiments, any or all components of the present invention further comprise a repository and/or retractable stage. These features allow for easy access to various instruments or medical items that be needed by a surgeon throughout a procedure, i.e., which are not traditionally within the custody of O.R. personnel. Such features of the invention are located on, within, or under any component of the adjustable ergonomic device, but in particular, the repository and/or retractable stage are associated with the platform section. Any or all components of the present invention may interact, connect, engage, lock, secure, rivet and/or disconnect, disengage, unlock, and remove from any or all components of the present invention in illustrative embodiments. Any or all components of the present invention are positioned in a manner selected from angled, straight, slanted, tapered, curved, diagonal, random, polygonal, rectangular, square, circular, curved, concentric, concave, perimetric, diamond, hexagonal, or triangular configurations, or any combination thereof capable of reversibly interacting, engaging, connecting, locking, and/or securing to any or all components of the present invention in suitable embodiments.

Such positioning, in some embodiments, secures via a plugging, screwing, capping, covering, sealing, corking, locking, riveting, and/or stopping mechanism, or any combination thereof. In suitable embodiments, all components are removable or interchangeable, or both. Moreover, all components may be configured as, e.g., hollow or solid rods, poles, members, tubes, plugs, screws, caps, rivets, covers, keys, wrenches, gripping members, and the like, or any combination thereof. In some embodiments, all components are composed of carbon fiber, silicone, rubber, vinyl, non-water hardenable urethane, plastic, plastic-based materials, fiberglass, metal, ceramic, polymers, resin, plaster, scented plastic, cellulose or any combination thereof, in suitable embodiments.

Various shapes and sizes of the adjustable ergonomic platforms are provided, and include, but are not limited to, for example, shapes such as, e.g., polygonal, rectangular, square, circular, curved, concentric, concave, perimetric, diamond, hexagonal, or triangular configurations, or any combination thereof The entire adjustable ergonomic platforms, but more specifically in some embodiments, the platform and/or platform surface is patterned, texturized, colorimetric, modifiable, or designable, or any combination thereof. Moreover, the present invention is fabricated or designed at the outset for a particular use in accord with the foregoing.

The design of the adjustable ergonomic device of the present disclosure provides for customized component materials and/or off the shelf components to be assembled prior to or in the O.R. Likewise, CAD software files and graphic representations of the present device allow for custom manufacturing to configure the device of the present technology for use in most any hospital setting for any procedure based on provided constraints and specifications. Nevertheless, all manufactured embodiments of the present invention are modifiable or adjustable to the user when performing a surgery. Another aspect of the present invention relates to rudimentary testing of a single unit prior to use at the point of care. Such initial testing includes, but is not limited to, simple bench testing and load testing with testing equipment. Because the unit demonstrates that it can likely withstand a pre-determined load, further modification can be avoided and/or performed based on a desired application.

As noted above, for increased comfort in using the arm rest aspect of the present invention, moreover, a silicone support pad is incorporated into an indented region of the arm rest region in some embodiments. In suitable embodiments, the supporting pad is place on the arm rest region for supporting the contacting regions of the surgeon's body. The supporting pad can be composed of various materials insofar as it improves comfort and stability of the user. Materials such as silicone, foam, rubber, vinyl, non-water hardenable urethane, plastic, plastic-based materials, fiberglass, metal, ceramic, polymers, resin, plaster, cellulosic materials or any combination thereof, in some embodiments, serve as the supporting pad materials. Such compositions allow for methodical placing, making, and/or circumferentially applying the supporting pad to various portions of the arm rest section. The indent within the arm rest for the supporting pad is subsequently used to design a custom mold to be manufactured using 3D printing or other conventional techniques, and to cast a supporting pad, e.g., silicone mat using a selected silicone resin in some embodiments.

FIGS. 3-4 is a side perspective view of one embodiment of a portable adjustable ergonomic device according to the present invention in an unfolded, operational configuration. The adjustable platform comprises a rigid support structure, such as a pair of collapsible sides, such as elongated horizontal beams, supported by a plurality of support members connected to and supporting the horizontal beams in certain embodiments. The plurality of support members are arranged in corresponding, opposing pairs and spaced apart along the length of the substantially parallel horizontal beams in certain embodiments.

When the platform is in use, opposing pairs or a single support members are connected to and rigidly braced apart by a pair of or a single pivotally connected cross-bar(s) held rigidly in place by means of a locking brace connected at either end of one or to both of the cross-bars, the locking brace or conduit can serve as a connecting link/supporting member with various uses. Such as conduit or connecting link may be pulled to straighten the one or more sections between the cross-bars and/or locking brace.

In another feature of the present invention, a platform attachment is provided with a bracing means. The bracing means comprises at least one beam affixed to the bottom surface of the platform/support board. Preferably, the beams extend from an inboard edge to and outboard edge, spanning the width of the platform in some embodiments. Likewise, in certain embodiments, the beams reinforce the weight carried by the platform when in use inasmuch as it supports the platform and stabilized the entire device to prevent vibrations. In illustrative embodiments, the beams are right angle beams, although other configurations can be used, such as I-beam, T-beam or box beam constructions. As with the platform/support, the beams are formed of sufficiently durable material, such as, but not limited to plastic or carbon fiber.

In some embodiments, the entire device/attachment is integrally formed as a unitary structure. In this embodiment, it may be molded from plastic, with the platform and conduits integral with the securement fixtures and accessory features. In other embodiments, the ergonomic device of the present invention is assembled of component sections to facilitate manufacture.

It should be appreciated from the descriptions of some embodiments herein that the ergonomic device of the present invention provides numerous functional and ergonomic benefits. The platform permits safe and comfortable positioning of the user's target region, which allows the surgeon and assistants to remain in a desire position during an O.R. procedure. The ergonomic device also provides a sturdy surface for supporting the humeral portion of the user's arm, i.e., the target region resting on the platform, in concert with additional retractable sections or attachments allowing for space to placing medical instruments. The platform in general stabilizes the upper or lower arm region of the user while allowing the other target regions to bend, e.g., at the elbow, and remain mobile. This positioning can prevent neurological damage to the ulnar nerve by preventing pressure points on the elbow.

### Methods

In one aspect, the present invention entails a method of preventing or reducing the occurrence of repetitive use injuries during a medical procedure, comprising: (a) providing an adjustable platform, wherein the adjustable platform is composed of a platform surface configured to conform to a body surface that defines a target region, wherein the surface is substantially contacted by one or more regions of a user's body; (b) adjusting the platform to a desired height by: (i) aligning the adjustable platform with a conduit, wherein the aligning allows for a cooperative interaction when substantially aligned; and securing one or more securement fixtures to the conduit, wherein the one or more securement fixtures are configured to reversibly engaged the one or more one or more conduits in an engaged configuration, and wherein the one or more securement fixtures are configured to reversibly disengaged the one or more conduits in a disengaged configuration; and (c) performing the medical procedure.

In illustrative embodiments, the medical procedure is selected from the group consisting of endoscopy, laparoscopy, surgery, minimally invasive surgery, and invasive surgery. In illustrative embodiments, the methods further include draping the adjustable platform. In some embodiments, the steps of aligning and/or securing comprises engaging the platform with the conduit at a locus when the conduit is in a vertical position, and wherein the securement fixture is engaged with the conduit at a second locus when the conduit is in a vertical positions. In illustrative embodiments, the desired height is based on the patient's positioning and the height of the surgeon performing the operation. In illustrative embodiments, the methods or adjustable ergonomic platforms of the present invention provide for a securement fixture that allows for attachment to the bed or operating table of the patient undergoing the procedure.

Such methods further entail, in suitable embodiments, securing the one or more securement fixtures 140 to the one or more conduits 130 and/or securing the one or more conduits 130 to the platform 110 by interacting, e.g., combining, the components to remain connected in some embodiments as shown in FIG. 2. In other embodiments, the securing is reversible. The securement fixtures are in a substantially aligned position with the one or more conduits 130 when in a secured position in some embodiments. Methods of the present invention further include securing the one or more securement fixtures 140 to the one or more conduits 130 such that the one or more fixtures and the one or more conduits 130 interact and remain connected when substantially aligned position with the one or more conduits 130 as shown in FIG. 2.

In some embodiments, the body surface is an entire body, lower extremity limbs, upper extremity limbs, one or both shoulders, one or both hips, one or both arms and/or forearms, one or both elbows, one or both hands, one or both wrists, one or both legs, one or both feet, one or both ankles, one or both heels, one or both knees, neck, and head, or any combination thereof. The target region is accordingly realized, which, in illustrative embodiments, includes, but is not limited to, for example, an entire body, lower extremity limbs, upper extremity limbs, one or both shoulders, one or both hips, one or both arms or forearms, one or both elbows, one or both hands, one or both wrists, one or both legs, one or both feet, one or both ankles, one or both heels, one or both knees, neck, and head, or any combination thereof. In suitable embodiment, the target region of the user is the arm or forearm region that contacts the platform section.

In some embodiments, one or more of the procedures, methods, steps, processes and the like, occur in about from 0.1-100, 0.5-90, 1-80, 1.5-70, 2-60, 2.5-55, 3-50, 3.5-40, 4-30, 4.5-20, 5-15, or 6-7 seconds minutes, hours, or days. In illustrative embodiments, they last from about 0.1-5 hours. It will be readily apparent to the skilled artisan that numerous additional variables can affect uses, methods, procedures and the like with respect to the present disclosure. These factors such as, for example, user-based factors include, but are not limited to general health, age, motivation, stress levels, mental fatigue, and performance under various empirical circumstances, while environmental considerations such as, but not limited to humidity, O₂ concentration, and/or temperature, etc., are contemplated, such that appropriate adjustments can optimize performance. For comfort of the user, clinician, or surgeon, foam edges or padding—in addition to the platform surface padding noted herein—may also be provided for any component of the present technology or on particularly troublesome regions, e.g., corners or edges that may be subject to contact with the user's target region. In this regard, two-part polyurethane foam available from Hastings Plastic Company or other materials which may be used are silicone, rubber, adhesive, foam, TPE, vinyl, non-water hardenable urethane, and other plastic materials, such as, for example, fiberglass, Kevlar®, or aramids, for examples, and to materials having comparable high strength, as discussed in U.S. Pat. Nos. 6,139,513, and 6,186,966. See, e.g., EP 1372549; See also EP 0755664.

Bonding material is also provided in some embodiments in the presence or absence of comfort foaming material or other applications as described above. The bonding material may be applied in a variety of ways, such as, for example, by spraying, brushing, pressing, dipping or the like. See, e.g., EP 1372549. Depending on the bonding material chosen and the method of application, it is possible that adjustments, manipulations, reconfigurations, and the like, can be performed, for example, for conforming to any and/or all components of the present invention, e.g., and/or placing the platform and/or platform surface or conduits therein, after application of the bonding material.

The methods and processes for using the device of the present invention entail both the substantive-physical use before and during the various surgical procedures, such as use in connection with an operating table, as disclosed herein, but also subsequent to the procedure, which includes steps relating to the cleaning, sterilization, assembly/disassembly and the like. In this respect, while the devices of the present invention are generally non-sterile insofar as a regulatory agency is concerned, the device must be cleaned and disinfected prior to any O.R. use using standard hospital techniques-process. The present technologies are in accord with such procedures inasmuch as they are compatible with hospital steam and EO sterilization processes. Nevertheless, prior to hospital sterilization, the various features of the present disclosure are packaged/wrapped with validated sterile barrier system, where the invention can accordingly be assembled at the point of surgery, i.e., the O.R. table using standard rail clamps, making certain all fittings are secure.

FDA Quality Systems Regulations (QSR) and CGMP requirements are also an important aspect of the present disclosure. While the present invention is a low-risk device (class 1 by FDA standards), good manufacturing practices must nevertheless be considered and followed throughout the manufacturing process. cGMP requirements include, but are not limited to, and thus, in some embodiments, the present invention comports with current good manufacturing practice (CGMP) requirements as set forth in the FDA quality system regulation, 21 C.F.R. §820. Such requirements concern the methods used in, and the facilities and controls used for, the design, manufacture, packaging, labeling, storage, installation, and servicing of all finished devices intended for human use including accessories. Due to the unexpected simplicity of the technology design, the regulatory requirements disclosed herein are satisfied to ensure that the present embodiments are safe and effective and otherwise in compliance with the Federal Food, Drug, and Cosmetic Act.

Along these lines, cleaning, disinfection and sterilization/re-sterilization of the present technologies, when required in the hospital setting, are regulatory aspects that are readily achieved in view of the straight-forward component system assembly even though such requirements and controls for cleaning, disinfection and terminal sterilization of re-usable medical devices are comprehensive and sometimes complicated process that can require an otherwise complex system multiple process validations. Moreover, the CDC (Centers of Disease Control) 2008 Guidance document, “Guideline for Disinfection and Sterilization in Healthcare Facilities” is recognized by the FDA. Within this CDC guidance document, the material defines a device, such as one or more embodiments disclosed herein, which comes in contact with only intact skin and non-direct patient contact as a “non-critical environmental surface” requiring a “reduced set” of processing.

In suitable embodiments, the cleaning, disinfecting and re-sterilization validation processes are coterminous with effective DFU/IFU (directions for use/instructions for use) that, in accord with the methods and uses disclosed herein, provide precise and validated instructions for such uses in accord with cleaning, disinfection and sterilization for re-usable medical devices. Some of the key standards and regulatory references which drive this process are as follows: FDA Guidance UCM252941—Reusable Device Reprocessing Resources for Health Care Facilities; CDC Guidance 2008—Guideline for Disinfection and Sterilization in Healthcare Facilities; AAMI TIR30_2011—A Compendium of Process, Materials, Test Methods, and Acceptance Criteria for Cleaning Reusable Medical Devices; AAMI TIR12_2010—Design Testing, and labeling Reusable Medical Devices for Reprocessing in Health Care Facilities: A Guide for Medical Device Manufacturers; and ANSI/AAMI ST79:2006—Comprehensive Guide to Steam Sterilization and Sterility Assurance in Health Care Facilities.

Accordingly, some embodiments of the present technologies are methods and uses that are performed in accordance with standard operating procedures (SOPs) to comply with the FDA quality system regulation, which also delineate directions/instructions for the intended uses within the O.R. to perform various surgical procedures such as, but not limited to, otolaryngic procedures (ear and laryngeal surgery) as well as general, gynecologic and urologic laparoscopic procedures, among other various endoscopic and laparoscopic surgeries. Likewise, within the scope of the present disclosure are process validation procedures to ensure that the assembly/use and cleaning, disinfecting and re-sterilization of the present technologies, among other embodiments and uses, are not only performed in the most efficient manner, but also modified within the general purview of the skilled artisan to maintain safety and efficacy.

Likewise, monitoring and validating the methods disclosed herein are within the scope of the present disclosure. In this respect, the ergonomic challenges that contribute to repetitive use injuries among endoscopic and laparoscopic surgeons are addressed and remedied by the present technology inasmuch as illustrative embodiments of the present disclosure relate to methods, uses, systems and products that eliminate or reduce injury risk in this regard. In some embodiments, a Field Test Acceptance protocol is developed and/or performed, where subsequent to completing one or more various surgeries, the surgeon and/or associated personnel will complete a questionnaire to ascertain and evaluate the most efficacious areas of functionality, ease of use, perceived and actual reduction in, for example, shoulder and neck strain, as well as and aesthetic appeal and portability. Similarly, in suitable embodiments, the efficacy of the present technology is confirmed and validate by determining the monetary impact of the present devices and uses thereof with respect to decreased time away from work at the individual (surgeons) and surgery center level over a predetermined period of time or procedures. The skilled artisan will readily appreciate that various metrics can be used for determining the overall impact of the present technology within a medical setting.

References: Ergonomic considerations in endoscopic sinus surgery: Lessons learned from laparoscopic surgeons. Ramakrishnan V, Montero; A m JRhinol Allergy, 245-25-, 2013; Ergonomics in the Operating Room: Protecting the Surgeon.Rosenblatt P, McKinney J, Adams S; Journal of Minimally Invasive Gynecology, 2013 (video article); Ergonomic principles associated with laparoscopic surgeon injury/illness. Miller K, Benden M, Pickens A, Shipp E, Zheng Q; Human Factors:The Journal of Human Factors and Ergonomics Society, 2012; (54)1087-1092; Patients Benefit While Surgeons Suffer: An ImpendingEpidemic. Park A, Lee G, Seagull F, Meenaghan N, Dexter D; J AM Coll Surg 2010;210:306-3013; Work-related Musculoskeletal Symptoms in Surgeons. Szeto G,Ho P, Ting A, Poon J, Cheng S, Tsang R; J Occup Rehabil 2009; 19:175-184; Optimal ergonomics for laparoscopic surgery in minimally invasive surgery suites: a review and guidelines. van Dat M, Meijerink W, HoffC, Totte E, Pierie J; Surg Endosc 2009; 23:1279-1285; Ergonomic considerations in endoscopic sinus surgery: Lessons learned frm laparoscopic surgeons. Ramakrishnan V, Montero; Am J Rhinol Allergy, 245-25-, 2013; Ergonomics in the Operating Room: Protecting the Surgeon. Rosenblatt P, McKinney J, Adams S; Journal of Minimally Invasinve Gyncology, 2013 (video article); Ergonomic principles associated with laparoscopic surgeon injury/illness. Miller K, Benden M, Pickens A, Shipp E, Zheng Q; Human Factors: The Journal of Human Factors and Ergonomics Society, 2012; (54)1087-1092; Patients Benefit While Surgeons Suffer: An Impending Epidemic. Park A, Lee G, Seagull F, Meenaghan N, Dexter D; J AM Coll Surg 2010; 210:306-3013; Work-related Musculoskeletal Symptoms in Surgeons. Szeto G, Ho P, Ting A, Poon J, Cheng S, Tsang R; J Occup Rehabil 2009; 19:175-184; and Optimal ergonomics for laparoscopic surgery in minimally invasive surgery suites: a review and guidelines. van Dat M, Meijerink W, Hoff C, Totte E, Pierie J; Surg Endosc 2009; 23:1279-1285.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 conduits refers to groups having 1, 2, or 3 conduits. Similarly, a group having 1-5 conduits refers to groups having 1, 2, 3, 4, or 5 conduits, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein are incorporated by reference herein in their entireties and for all purposes to the same extent as if each individual publication, patent, or patent application was specifically and individually incorporated by reference in its entirety for all purposes.

1. An adjustable ergonomic platform, comprising:
(a) the adjustable platform;
(b) a platform surface configured to conform to a body surface that defines a target region, wherein the surface is substantially contacted by one or more regions of a user's body;
(c) one or more conduits capable of cooperatively interacting with the adjustable platform when each component is substantially aligned; and
(d) one or more securement fixtures, wherein the one or more securement fixtures are configured to reversibly engaged the one or more one or more conduits in an engaged configuration, and wherein the one or more securement fixtures are configured to reversibly disengaged the one or more conduits in a disengaged configuration.

2. The adjustable ergonomic platform of claim 1, further comprising one or more of drapes, power generating pedals, venting components and/or padding.

3. The adjustable ergonomic platform of claim 1, wherein the conduit is in an orthogonal position relative to the platform.

4. The adjustable ergonomic platform of claim 1, wherein the conduit is a vertical rail, wherein the platform is capable of sliding along the rail when engaged.

5. The adjustable ergonomic platform of claim 1, wherein the platform is engaged with the conduit at a locus when the conduit is in a vertical position, and wherein the securement fixture is engaged with the conduit at a second locus when the conduit is in a vertical positions.

6. The adjustable ergonomic platform of claim 1, wherein the platform surface is composed of a material selected from the group consisting of silicone, rubber, vinyl, non-water hardenable urethane, plastic, plastic-based materials, fiberglass, metal, ceramic, one or more polymers, resin, plaster, scented plastic, cellulose, and scented cellulose, silicone, rubber, vinyl, non-water hardenable urethane, plastic, plastic-based materials, fiberglass, metal, ceramic, polymers, resin, plaster, scented plastic, cellulose, or scented cellulose, or any combination thereof.

7. The adjustable ergonomic platform of claim 1, wherein the target region is a body surface is selected from the group consisting of one or both forearms, one or both arms, an entire body, lower extremity limbs, upper extremity limbs, one or both shoulders, one or both hips, one or both arms, one or both elbows, one or both hands, one or both wrists, one or both legs, one or both feet, one or both ankles, one or both heels, one or both knees, neck, and head, or any combination thereof.

8. The adjustable ergonomic platform of claim 1, wherein the platform has a shape selected from the group consisting of circular, rectangular, square, polygonal, curved, concentric, concave, perimetric, diamond, hexagonal, or triangular configurations, or any combination thereof.

9. The adjustable ergonomic platform of claim 1, wherein the platform and/or platform surface allows for treatment, medicament application, topical cream application, application of ointments, cold compresses application, application of ice, coolant application, or for applying heat, or any combination thereof.

10. The adjustable ergonomic platform of claim 1, wherein the height of the adjustable ergonomic platform is about from 0.01 m to about 10 m.

11. The adjustable ergonomic platform of claim 1, wherein the one or more conduits and/or the securement fixtures are composed of silicone, rubber, vinyl, non-water hardenable urethane, plastic, plastic-based materials, fiberglass, metal, ceramic, one or more polymers, resin, plaster, scented plastic, cellulose, and scented cellulose, or any combination thereof.

12. The adjustable ergonomic platform of claim 1, wherein the platform and/or the platform surface allows for ventilation, moisture release, liquid drainage, or drying, or any combination thereof.

13. The adjustable ergonomic platform of claim 1, wherein the one or more securement fixtures are selected from the group consisting of plugs, screws, caps, covers, seals, corks, rivets, and stoppers, or any combination thereof.

14. The adjustable ergonomic platform of claim 1, wherein the conduit and/or the one or more securement fixtures interact with the platform and/or one or more conduits in a manner selected from the group consisting of angled, straight, slanted, tapered, orthogonal, polygonal, curved, diagonal, random, or any combination thereof.

15. The adjustable ergonomic platform of claim 1, wherein the platform, platform surface, one or more conduits and/or securement fixtures are removable or interchangeable, or both.

16. (canceled)

17. The adjustable ergonomic platform of claim 1, wherein the adjustable ergonomic platform further comprises a repository.

18. (canceled)

19. (canceled)

20. (canceled)

21. (canceled)

22. (canceled)

23. A method of preventing or reducing the occurrence of repetitive use injuries during a medical procedure, comprising:
(a) providing an adjustable platform, wherein the adjustable platform is composed of a platform surface configured to conform to a body surface that defines a target region, wherein the surface is substantially contacted by one or more regions of a user's body;
(b) adjusting the platform to a desired height by:
aligning the adjustable platform with a conduit, wherein the aligning allows for a cooperative interaction when substantially aligned; and
(ii) securing one or more securement fixtures to the conduit, wherein the one or more securement fixtures are configured to reversibly engaged the one or more one or more conduits in an engaged configuration, and wherein the one or more securement fixtures are configured to reversibly disengaged the one or more conduits in a disengaged configuration; and
(c) performing the medical procedure.

24. The method of claim 23, wherein the medical procedure is selected from the group consisting of endoscopy, laparoscopy, surgery, minimally invasive surgery, and invasive surgery.

25. The method of claim 23 further comprising one or more of draping the adjustable platform.

26. The method of claim 23, wherein the aligning and/or securing comprises engaging the platform with the conduit at a locus when the conduit is in a vertical position, and wherein the securement fixture is engaged with the conduit at a second locus when the conduit is in a vertical positions.

27. (canceled)

28. (canceled)
